(19) United States
(12) Patent Application Publication
(10) Pub. No.: US 2009/0325221 A1
(43) Pub. Date: Dec. 31, 2009

(54) Temporary Tattoo Decals for Detecting the Presence of an Analyte

(75) Inventors: Andrew Mark Long, Appleton, WI (US); John Gavin MacDonald, Decatur, GA (US); Tom Michael Ales, III, Neenah, WI (US); Lindsey Marie Jain, Winona Lake, IN (US)

Correspondence Address: DORITY & MANNING, P.A., POST OFFICE BOX 1449, GREENVILLE, SC, 29602-1449, US

(73) Assignee: Kimberly-Clark Worldwide, Inc., Neenah, WI (US)

(21) Appl. No.: 12/164,450

(22) Filed: Jun. 30, 2008

(51) Int. Cl. C12Q 1/04; C12M 1/34

(52) U.S. Cl. 435/34; 435/287.9

(57) ABSTRACT

Temporary tattoos to be applied to the skin of a wearer for the detection of an analyte are generally disclosed. The temporary tattoo can be applied to the skin via a temporary tattoo decal. The temporary tattoo can indicate the presence of an analyte by displaying a certain spectral response (e.g., color change) in the presence of a targeted analyte. This change in color can indicate to the wearer and/or caregiver the presence of the targeted analyte in real-time.

### BACKGROUND OF THE INVENTION

Information about the presence of contaminants (e.g., chemicals, microbes, and other analytes) surrounding or on a person's skin can be an invaluable tool to recognize a potential threat to that person's health and wellbeing. Once the presence of such a contaminant is recognized, then proper measures can be taken to address the contaminant within the environment.

Many different types of chemical sensing devices can be utilized to detect the presence of such contaminants. For example, chemical sensors can be placed within a building to monitor the presence of a contaminant within the air. Also, chemical sensing strips can be utilized to detect the presence of an analyte within a test sample (e.g., a liquid or gas).

However, it is often desired to monitor the environment of a person only on a temporary basis. As such, a need exists for disposable diagnostic tools that quickly and reliably detect the presence of an analyte and alert the person or a caregiver.

### SUMMARY OF THE INVENTION

Objects and advantages of the invention will be set forth in part in the following description, or may be obvious from the description, or may be learned through practice of the invention.

In general, the present disclosure is directed toward a temporary tattoo decal for application to a wearer's skin and configured to detect the presence of an analyte. The temporary tattoo decal can include a base paper, a water-soluble slip layer, a temporary tattoo, an adhesive layer, and a protective sheet. The water-soluble slip layer can be applied to the base paper to allow for the release of the base paper from the temporary tattoo upon contact with water (or another appropriate solvent). The temporary tattoo can be applied to the water-soluble release layer on the base paper in the form of an image. The temporary tattoo includes a chemical indicator configured to change color upon contact with the analyte. An adhesive layer overlies the temporary tattoo, and a protective sheet overlying the adhesive layer.

The present invention is also directed to, in another embodiment, a method of detecting the presence of an analyte. The method includes first applying a temporary tattoo to skin of a wearer. The temporary tattoo forms an image and includes a chemical indicator configured to change color upon contact with the analyte. Then, the temporary tattoo can be monitored to determine if a color change has occurred. If such a color change is observed, then the presence of the analyte can be reported.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures, in which:

FIG. 1 shows an exemplary temporary tattoo decal according to one embodiment of the present invention;

FIGS. 2-4 sequentially show an exemplary method of applying the temporary tattoo decal of FIG. 1 to the skin.

Repeat use of reference characters in the present specification and drawings is intended to represent the same or analogous features or elements of the present invention.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

In general, the present disclosure is directed to temporary tattoos that can be applied to the skin of a wearer. In one embodiment, the temporary tattoo can be applied to the skin via a temporary tattoo decal. Once applied, the temporary tattoo can detect the presence of an analyte and alert the wearer or a caregiver. Thus, the person or caregiver can take appropriate action to address the presence of the analyte. Additionally, the temporary tattoo can be removed from the skin (e.g., washed away) once the period of testing has expired or can simply fade away after a period of time (e.g., 2-5 days).

The temporary tattoo can indicate the presence of an analyte by displaying a certain spectral response (e.g., color change) in the presence of a targeted analyte. For example, the temporary tattoo may change from a first color to a second color, from colorless to a color, or from a color to colorless. This change in color can indicate to the wearer and/or caregiver the presence of the targeted analyte in real-time. As used herein, the term “analyte” refers to any chemical compound, element, microorganism, or other substance which can be a gas, vapor, liquid, or solid phase.

According to the present invention, temporary tattoo decals can be utilized to quickly and efficiently apply a temporary tattoo to a surface (typically skin of a wearer) in order to detect the presence of an analyte and alert the wearer or caregiver. The temporary tattoo can form an image on the surface to provide an aesthetic quality to the temporary tattoo. The image formed by the temporary tattoo can form any shape, character, or other artwork desired. For example, the image can form one or more characters (e.g., cartoon character, alphanumeric characters, etc.) that can be appealing to the wearer, especially when the wearer is a child.

### I. Temporary Tattoo Decals

The temporary tattoo can be applied to the skin of a wearer via a temporary tattoo decal. In this embodiment, the decal can be pre-manufactured and readily available to be applied to the skin or other surface. The temporary tattoo decals also provide a relatively easy method of applying the temporary tattoo to the skin of the wearer.

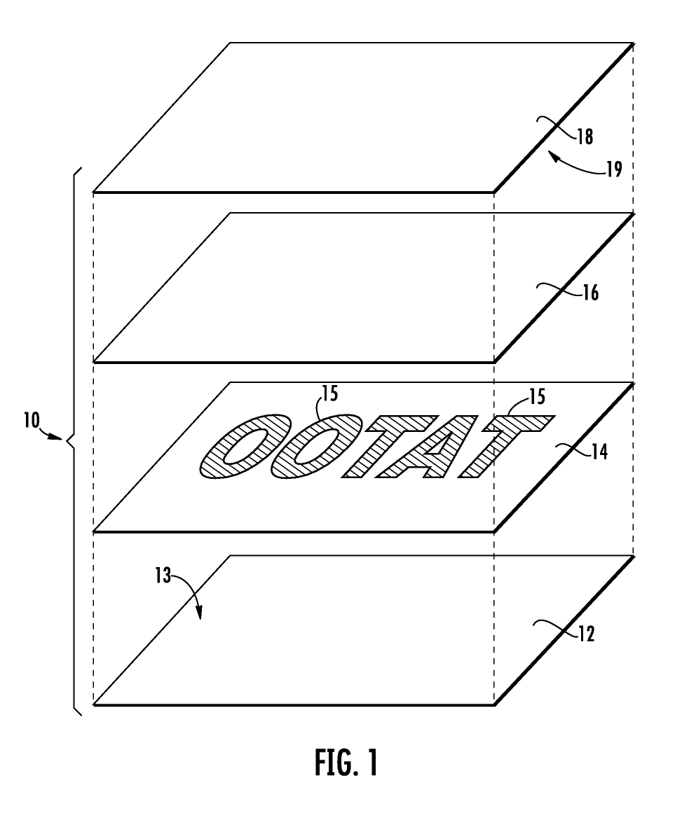

In one particular embodiment, the temporary tattoo decal includes a base paper, a water-soluble slip layer, the temporary tattoo, an adhesive layer, and a protective sheet. For example, referring to FIG. 1, an exemplary temporary tattoo decal 10 is shown. The temporary tattoo decal 10 includes a base paper 12 that acts primarily to provide a support structure for the decal 10. The base paper 12 can be made according to any method of paper making. Typically, the base paper 12 can be made from a random plurality of papermaking fibers (e.g., cellulosic fibers) that can, optionally, be joined together with a binder. Of course, other materials can be present within the base paper 12 as desired, such as synthetic polymeric fibers, colorants, filler material, etc.

A water-soluble slip layer 14 overlies the base paper 12. Although shown as a separate layer for the purposes of illustration in FIG. 1, the water-soluble slip layer 14 may be a coating applied directly to the top surface 13 of the base paper 12. For example, the water-soluble slip layer 14 may be a film or nonwoven web formed directly on the top surface 13 of the base paper 12. The water-soluble slip layer 14 is configured to dissolve upon contact with water or another solvent. For example, the water-soluble slip layer 14 can be soluble or dispersible in water, alcohol, oil or another reagent that is safe for skin contact.

The water-soluble slip layer 14 essentially holds the base paper to the temporary tattoo to form the decal. However, upon contact with water (or another reagent), the water-soluble slip layer 14 can disperse and allow for the release of the base paper 12 from the adhesive layer 16 and temporary tattoo 15. This step completes the transfer of the temporary tattoo from the decal to the skin of the wearer.

The water-soluble slip layer 14 can include, in one particular embodiment, a water-soluble polymeric material in the form of a film or a nonwoven web. In some embodiments, the water-soluble polymeric material can include, but is not limited to, polyvinyl alcohol, sodium alginate, hydroxypropyl methylcellulose, chitosan, polyethylene glycol, tetramethylene ether glycol, polyvinyl pyrrolidone, hydroxymethyl cellulose, and combinations thereof. However, any suitable water-soluble material can be utilized to form the water-soluble slip layer 14.

The temporary tattoo 15 can be formed on the exposed surface of the water-soluble slip layer 14, which is the surface opposite the base paper 12. In FIG. 1, the temporary tattoo 15 is shown using alphanumberic characters, although any image can be utilized to form the tattoo, as stated above. Also, the image is a mirror image of that to be applied to the skin due to the manner in which the decal 10 is applied, as explained in greater detail below.

Overlying the temporary tattoo 15 is an adhesive layer 16 and a protective sheet 18. The adhesive layer 16 is configured to stick to the skin or other surface to which the temporary tattoo is to be applied. The adhesive layer can be applied directly over the temporary tattoo 15 and water-soluble slip layer 14 or can be applied to the back surface 19 of the protective sheet 18. In either application, the adhesive layer 16 is positioned between the temporary tattoo 15 and the protective sheet 18.

In one embodiment, both the adhesive layer 16 and the protective sheet 18 are substantially transparent such that the underlying image formed by the temporary tattoo can be viewed through the layers. Additionally, a transparent adhesive layer will not be visible on the skin of the wearer. The adhesive layer 16 can be any glue or other tacky material that is safe for application to the skin.

The protective sheet 18 acts primarily to cover and protect the adhesive layer 16 until removed to expose the underlying adhesive layer 16 for application to the targeted area. The protective sheet 18 can be a film, nonwoven web, or other flexible sheet. In one particular embodiment, the protective sheet 18 is a transparent film that enables the underlying image formed by the temporary tattoo 15 to be viewed. Suitable transparent films can be formed from any transparent polymeric material capable of forming a flexible film, such as polyolefins (e.g., polyethylene, polypropylene, etc), polyesters (e.g., polyethylene terephthalate), polyvinylidene chloride, polyvinyl acetates, and copolymers and combinations thereof.

In one particular embodiment, when the temporary tattoo is applied via a decal, the temporary tattoo is formed by applying a chemical indicator composition to the base paper in the form of the desired image to be transferred. However, the image is formed on the base paper as a mirror image of that to eventually be found on the skin, due to the transfer method. Any suitable application method can be utilized to form the image on the base paper including, but not limited to, printing, dipping, spraying, melt extruding, coating (e.g., solvent coating, powder coating, brush coating, etc.), spraying, and so forth. Printing techniques may include, for instance, gravure printing, flexographic printing, screen printing, laser printing, thermal ribbon printing, piston printing, etc.

The chemical indicator composition includes the chemical indicator and any other desirable components. In one embodiment, the chemical indicator composition may be formed as a printing ink using any of a variety of known components and/or methods. For example, the printing ink may contain water as a carrier, and particularly deionized water. Various co-carriers may also be included in the ink, such as lactam, N-methyl pyrrolidone, N-methylacetamide, N-methylmorpholine-N-oxide, N,N-dimethylacetamide, N-methyl formamide, propyleneglycol-monomethylether, tetramethylene sulfone, tripropyleneglycolmonomethylether, propylene glycol, and triethanolamine (TEA). Humectants may also be utilized, such as ethylene glycol; diethylene glycol; glycerine; polyethylene glycol 200, 300, 400, and 600; propane 1,3 diol; propylene-glycolmonomethyl ethers, such as Dowanol PM (Gallade Chemical Inc., Santa Ana, Calif.); polyhydric alcohols; or combinations thereof.

The exact quantity of the chemical indicator employed within the chemical indicator composition may vary based on a variety of factors, including the sensitivity of the indicator, the presence of other additives, the desired degree of detectability (e.g., with an unaided eye).

### II. Application to the Skin of a Wearer

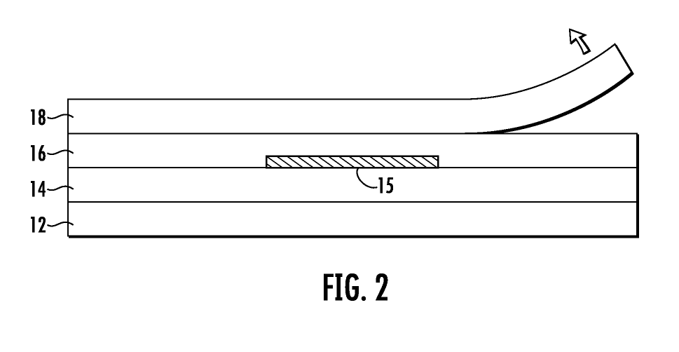

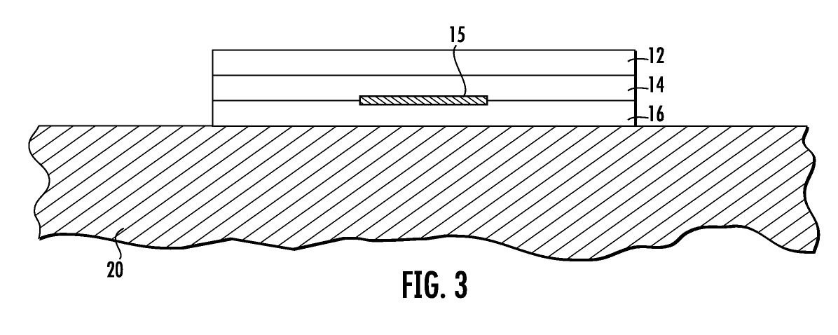

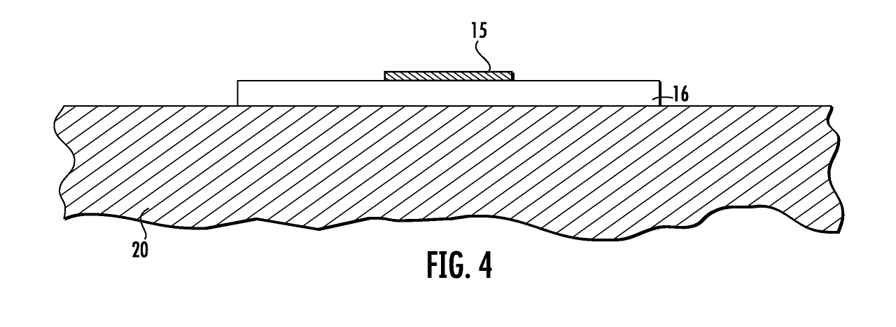

In order to transfer the temporary tattoo 15 to the skin of a wearer from a temporary tattoo decal, the protective sheet 18 is first peeled from the decal 10 to reveal the underlying adhesive layer 16, as shown in FIG. 2. The adhesive layer 16 can then be positioned adjacent to the skin 20, as shown in FIG. 3. Thus, the temporary tattoo 15 is adhered to the skin 20 via the adhesive layer 16.

Finally, the base paper 12 can be moistened with water (or another appropriate solvent). The solvent saturates the base paper 12, due to its hydrophilic nature, and penetrates through the base paper 12 to contact the underlying water-soluble slip layer 14. The solvent can then solubilize or disperse the water-soluble slip layer 14 allowing the base paper 12 to be released. Thus, the temporary tattoo 15 is left adhered to the skin 20 of the wearer.

Of course, other layers and configurations can be utilized to form the temporary tattoo 15 on the skin of the wearer. For example, the temporary tattoo can be applied to the skin with brushes and/or pens or similar implements in a fashion analogous to making an illustration on paper. In this embodiment, a stencil can be used to form the image or the image may be drawn free-hand.

### III. Chemical Indicators

As stated, the chemical indicator is applied to the skin of the wearer in the form of a temporary tattoo. No matter the application method, the temporary tattoo of the present invention includes a chemical indicator that is configured to display a certain spectral response (e.g., color change) in the presence of a targeted analyte. For example, the temporary tattoo may change from a first color to a second color, from colorless to a color, or from a color to colorless. This change in color can indicate to the wearer and/or caregiver the presence of the targeted analyte in real-time.

In one embodiment, the temporary tattoo can be applied as a clear image, which will only appear upon contact with a microorganism. Thus, the temporary tattoo can be substantially invisible on the exposed skin of the wearer, but still able to detect the presence of the targeted analyte.

A temporary tattoo according to the invention may also be produced so that certain designated areas of the artwork contain different chemical indicators within the same image.

The chemical indicator can be any material that changes color upon contact with a certain analyte. Many such chemical indicators can be particular to a particular analyte or class of analytes. For example, acetone hydrazine or ketonuria acetoacetic acid nitroprusside can be used as a ketone chemical indicator to detect the presence of ketones. When a ketone is present, this ketone chemical indicator changes from colorless to blue. A ketone can be defined as a chemical compound having a carbonyl group (O═C) linked to two other carbon atoms.

In another embodiment, ferric chloride, 4-amino-antipyrine, and/or 2,6-dibromoquinone-4-chlorimide can be used as a chemical indicator to detect poison ivy, or more specifically the urushiol that is a series of alkyl-substitued catechols that comprise the oil causing the blisters and rashes. Upon contact with poison ivy (Poison ivy (Rhus radicans), poison oak (Rhus toxicodendron) and poison sumac (Rhus vernix)), the chemical indicator within the temporary tattoo can change from colorless to blue.

In yet another embodiment, the chemical indicator can be 4,4′-bis(dimethylamino)-benzhydrol, also known as “BDMD”, “Michler's hydrol” or “MH”. This indicator reacts with amine or sulfur compounds and is sensitive to both sulfur-containing and ammonia-containing odors, changing from blue to colorless in the presence of these odors.

Several other suitable chemical indicators are discussed below in greater detail. However, it should be understood that the present invention is not limited to any particular type of chemical indicator.

A. Chemical Indicators for Bacteria

In one particular embodiment, the chemical indicator employed in the temporary tattoo of the present invention can provide a broad spectrum response for bacteria or other microorganisms. This embodiment can be particularly useful as a deterrent for spreading infections in heavily populated buildings that have been susceptible to spreading germs in the past (e.g., hospitals, schools, public buildings, etc.). In this embodiment, the temporary tattoo 15 can be applied to the hand or forearm of the wearer (or another exposed area of skin on the wearer). Thus, the temporary tattoo can alert the wearer when he or she has contacted a microorganism. Such an early detection method can help prevent the spread of infections and other microorganism related health problems.

The response for bacteria or other microorganisms can be the same or different than its response for viruses. For example, solvatochromatic indicators are particularly effective in undergoing a distinct color change in the presence of a broad spectrum of bacteria or other microorganisms, yet very little if any change in the presence of viruses associated with upper respiratory conditions.

Merocyanine indicators (e.g., mono-, di-, and tri-merocyanines) are one example of a type of solvatochromatic indicator that may be employed in the present invention. Merocyanine indicators, such as merocyanine 540, fall within the donor—simple acceptor indicator classification of Griffiths as discussed in “Colour and Constitution of Organic Molecules” Academic Press, London (1976). More specifically, merocyanine indicators have a basic nucleus and acidic nucleus separated by a conjugated chain having an even number of methine carbons. Such indicators possess a carbonyl group that acts as an electron acceptor moiety. The electron acceptor is conjugated to an electron donating group, such as a hydroxyl or amino group. The merocyanine indicators may be cyclic or acyclic (e.g., vinylalogous amides of cyclic merocyanine indicators). Merocyanine indicators typically have a charge separated (i.e., “zwitterionic”) resonance form. Zwitterionic indicators are those that contain both positive and negative charges and are net neutral, but highly charged. Without intending to be limited by theory, it is believed that the zwitterionic form contributes significantly to the ground state of the indicator. The color produced by such indicators thus depends on the molecular polarity difference between the ground and excited state of the indicator.

Indigo is another example of a suitable solvatochromatic indicator for use in the present invention. Indigo has a ground state that is significantly less polar than the excited state.

Other suitable solvatochromatic indicators that may be used in the present invention include those that possess a permanent zwitterionic form. That is, these indicators have formal positive and negative charges contained within a contiguous π-electron system. Exemplary indicators of this class include N-phenolate betaine indicators.

Reichardt's dye shows strong negative solvatochromism and may thus undergo a significant color change from blue to colorless in the presence of bacteria. That is, Reichardt's dye displays a shift in absorbance to a shorter wavelength and thus has visible color changes as solvent eluent strength (polarity) increases.

Still other suitable solvatochromatic indicators may include, but are not limited to 4-dicyanmethylene-2-methyl-6-(p-dimethylaminostyryl)-4H-pyran (DCM); 6-propionyl-2-(dimethylamino)naphthalene (PRODAN); 9-(diethylamino)-5H-benzo[a]phenox-azin-5-one (Nile Red); 4-(dicyanovinyl)julolidine (DCVJ); phenol blue; stilbazolium indicators; coumarin indicators; ketocyanine indicators; N,N-dimethyl-4-nitroaniline (NDMNA) and N-methyl-2-nitroaniline (NM2NA); Nile blue; 1-anilinonaphthalene-8-sulfonic acid (1,8-ANS), and dapoxylbutylsulfonamide (DBS) and other dapoxyl analogs. Besides the above-mentioned indicators, still other suitable indicators that may be used in the present invention include, but are not limited to, 4-[2-N-substituted-1,4-hydropyridin-4-ylidine)ethylidene]cyclohexa-2,5-dien-1-one, red pyrazolone indicators, azomethine indicators, indoaniline indicators, and mixtures thereof. Various suitable solvatochromatic colorants that are suitable for use in the present invention are described in U.S. Patent Application Publication No. 2006/0134728 to MacDonald, et al., which is incorporated herein in its entirety by reference thereto for all purposes.

In addition to a broad spectrum indicator, one or more indicators (e.g., dyes, pigments, etc.) can also be employed that are capable of differentiating between certain types of microorganisms. pH-sensitive indicators, for instance, may be employed that can detect a change in the pH of the growth medium of the microorganism. Bacteria and viruses, for instance, may metabolize the growth medium and generate acidic compounds (e.g., CO2) or basic compounds (e.g., ammonia) that lead to a change in pH. Likewise, certain microorganisms (e.g., bacteria) contain highly organized acid moieties on their cell walls. Because the acidic/basic shift may vary for different microorganisms, pH-sensitive indicators may be selected in the present invention that are tuned for the desired pH transition. In this manner, the temporary tattoo may be provided with pH-sensitive indicators that are configured to undergo a detectable color change only in the presence of bacteria or viruses exhibiting a certain acidic/basic shift.

Phthalein indicators constitute one class of suitable pH-sensitive indicators that may be employed in the temporary tattoo of the present invention. Phenol Red (i.e., phenolsulfonephthalein), for example, exhibits a transition from yellow to red over the pH range 6.6 to 8.0. Above a pH of about 8.1, Phenol Red turns a bright pink (fuschia) color. Derivatives of Phenol Red may also be suitable for use in the present invention, such as those substituted with chloro, bromo, methyl, sodium carboxylate, carboxylic acid, hydroxyl and amine functional groups. Exemplary substituted Phenol Red compounds include, for instance, Chlorophenol Red, Metacresol Purple (meta-cresolsulfonephthalein), Cresol Red (ortho-cresolsulfonephthalein), Pyrocatecol Violet (pyrocatecolsulfonephthalein), Chlorophenol Red (3′,3″-dichlorophenolsulfonephthalein), Xylenol Blue (the sodium salt of para-xylenolsulfonephthalein), Xylenol Orange, Mordant Blue 3 (C.I. 43820), 3,4,5,6-tetrabromophenolsulfonephthalein, Bromoxylenol Blue, Bromophenol Blue (3′,3″,5′,5″-tetrabromophenolsulfonephthalein), Bromochlorophenol Blue (the sodium salt of dibromo-5′,5″-dichlorophenolsulfonephthalein), Bromocresol Purple (5′,5″-dibromo-ortho-cresolsulfonephthalein), Bromocresol Green (3′,3″,5′,5″-tetrabromo-ortho-cresolsulfonephthalein), and so forth. Still other suitable phthalein indicators are well known in the art, and may include Bromothymol Blue, Thymol Blue, Bromocresol Purple, thymolphthalein, and phenolphthalein (a common component of universal indicators). For example, Chlorophenol Red exhibits a transition from yellow to red over a pH range of about 4.8 to 6.4; Bromothymol Blue exhibits a transition from yellow to blue over a pH range of about 6.0 to 7.6; thymolphthalein exhibits a transition from colorless to blue over a pH range of about 9.4 to 10.6; phenolphthalein exhibits a transition from colorless to pink over a pH range of about 8.2 to 10.0; Thymol Blue exhibits a first transition from red to yellow over a pH range of about 1.2 to 2.8 and a second transition from yellow to pH over a pH range of 8.0 to 9.6; Bromophenol Blue exhibits a transition from yellow to violet over a pH range of about 3.0 to 4.6; Bromocresol Green exhibits a transition from yellow to blue over a pH range of about 3.8 to 5.4; and Bromocresol Purple exhibits a transition from yellow to violet over a pH of about 5.2 to 6.8.

Hydroxyanthraquinones constitute another suitable class of pH-sensitive indicators for use in the present invention. Hydroxyanthraquinones have a fused ring structure at which substitution of a functional group may occur. For hydroxyanthraquinones, at least one of the functional groups is or contains a hydroxy (—OH) group. Other examples of functional groups that may be substituted on the fused ring structure include halogen groups (e.g., chlorine or bromine groups), sulfonyl groups (e.g., sulfonic acid salts), alkyl groups, benzyl groups, amino groups (e.g., primary, secondary, tertiary, or quaternary amines), carboxy groups, cyano groups, phosphorous groups, etc. Some suitable hydroxyanthraquinones that may be used in the present invention, Mordant Red 11 (Alizarin), Mordant Red 3 (Alizarin Red S), Alizarin Yellow R, Alizarin Complexone, Mordant Black 13 (Alizarin Blue Black B), Mordant Violet 5 (Alizarin Violet 3R), Alizarin Yellow GG, Natural Red 4 (carminic acid), amino-4-hydroxyanthraquinone, Emodin, Nuclear Fast Red, Natural Red 16 (Purpurin), Quinalizarin, and so forth. For instance, carminic acid exhibits a first transition from orange to red over a pH range of about 3.0 to 5.5 and a second transition from red to purple over a pH range of about 5.5 to 7.0. Alizarin Yellow R, on the other hand, exhibits a transition from yellow to orange-red over a pH range of about 10.1 to 12.0.

Arylmethanes (e.g., diarylmethanes and triarylmethanes) constitute still another class of suitable pH-sensitive indicators for use in the present invention.

Still other suitable pH-sensitive indicators that may be employed in the test strip include Congo Red, Litmus (azolitmin), Methylene Blue, Neutral Red, Acid Fuchsin, Indigo Carmine, Brilliant Green, Picric acid, Metanil Yellow, m-Cresol Purple, Quinaldine Red, Tropaeolin OO, 2,6-dinitrophenol, Phloxine B, 2,4-dinitrophenol, 4-dimethylaminoazobenzene, 2,5-dinitrophenol, 1-Naphthyl Red, Chlorophenol Red, Hematoxylin, 4-nitrophenol, nitrazine yellow, 3-nitrophenol, Alkali Blue, Epsilon Blue, Nile Blue A, universal indicators, and so forth. For instance, Congo Red undergoes a transition from blue to red at a pH range of about 3.0 to 5.2, Litmus undergoes a transition from red to blue at a pH range of about 4.5 to 8.3, and Neutral Red undergoes a transition from red to yellow at a pH range of about 11.4 to 13.0.

In addition to pH, other mechanisms may also be wholly or partially responsible for inducing a color change in the indicators. For example, many microorganisms (e.g., bacteria) produce low molecular weight iron-complexing compounds in growth media, which are known as “siderophores.” Metal complexing indicators may thus be employed in some embodiments of the present invention that undergo a color change in the presence of siderophores. One particularly suitable class of metal complexing indicators are aromatic azo compounds, such as Eriochrome Black T, Eriochrome Blue SE (Plasmocorinth B), Eriochrome Blue Black B, Eriochrome Cyanine R, Xylenol Orange, Chrome Azurol S, carminic acid, etc. Still other suitable metal complexing indicators may include Alizarin Complexone, Alizarin S, Arsenazo III, Aurintricarboxylic acid, 2,2′-Bipyidine, Bromopyrogallol Red, Calcon (Eriochrome Blue Black R), Calconcarboxylic acid, Chromotropic acid, disodium salt, Cuprizone, 5-(4-Dimethylamino-benzylidene)rhodanine, Dimethylglyoxime, 1,5-Diphenylcarbazide, Dithizone, Fluorescein Complexone, Hematoxylin, 8-Hydroxyquinoline, 2-Mercaptobenzothiazole, Methylthymol Blue, Murexide, 1-Nitroso-2-naphthol, 2-Nitroso-1-naphthol, Nitroso-R-salt, 1,10-Phenanthroline, Phenylfluorone, Phthalein Purple, 1-(2-Pyridylazo)-naphthol, 4-(2-Pyridylazo)resorcinol, Pyrogallol Red, Sulfonazo III, 5-Sulfosalicylic acid, 4-(2-Thiazolylazo)resorcinol, Thorin, Thymolthalexon, Tiron, Tolurnr-3,4-dithiol, Zincon, and so forth. It should be noted that one or more of the pH-sensitive indicators referenced above may also be classified as metal complexing indicators.

Although the above-referenced indicators are classified based on their mechanism of color change (e.g., pH-sensitive, metal complexing, or solvatochromatic), it should be understood that the present invention is not limited to any particular mechanism for the color change. Even when a pH-sensitive indicator is employed, for instance, other mechanisms may actually be wholly or partially responsible for the color change of the indicator. For example, redox reactions between the indicator and microorganism may contribute to the color change.

As a result of the present invention, it has been discovered that the presence of bacteria, viruses, or other microorganisms may be readily detected through the use of indicators that undergoes a detectable color change. The color change is rapid and may be detected within a relatively short period of time. For example, the change may occur in about 30 minutes or less, in some embodiments about 10 minutes or less, in some embodiments about 5 minutes or less, in some embodiments about 3 minutes or less, and in some embodiments, from about 10 seconds to about 2 minutes. In this manner, the indicator may provide a “real-time” indication of the presence or absence of microorganisms. Such a “real time” indication may alert a user or caregiver to seek treatment (e.g., antibiotic). On the other hand, the lack of a certain color change may provide the user or caregiver with an assurance that the sample is free of infection.

B. Chemical Indicators for Yeast

In another embodiment, the temporary tattoo can be configured to detect the presence of yeast, such as Candida albicans. One of the more problematic secondary infections associated with diaper rash is “yeast infection”, which is typically caused by Candida albicans. Under the conditions that result in diaper rash, for instance, the normally unicellular yeast-like form of Candida albicans can convert into an invasive, multicellular filamentous form. Candida albicans may result in painful swelling and become difficult to resolve.

In this embodiment, the temporary tattoo of the present invention can be applied to the skin of a wearer that is covered by the diaper. Thus, the caregiver can easily tell if Candida albicans is present within the diaper upon changing of the diaper.

The chemical indicator included within the temporary tattoo can be capable of differentiating between Candida (e.g., Candida albicans) and other microorganisms commonly associated with diaper rash, such as S. aureus and E. coli. Thus, when the temporary tattoo is positioned on the skin of the wearer, the color change may simply be observed to determine whether the infection is caused by Candida. If the color change occurs to a certain extent (e.g., from yellow to bright red), it may be determined that the test sample contains Candida. Likewise, if a color change occurs to a lesser extent (e.g., from yellow to faint orange) or not at all, it may be determined that other other microorganisms (e.g., S. aureus or E. coli) is present on the skin, no infection is present, or that the infection is simply due to other causes. Regardless, it will become readily apparent whether or not treatment for Candida is needed.

One particularly suitable class of colorants that may undergo a detectable color change in the presence of Candida is pH-sensitive colorants. Namely, pH-sensitive colorants can detect a change in the pH of the growth medium of the microorganism. Because the acidic/basic shift may vary for different microorganisms, pH-sensitive colorants may be selected that are tuned for the desired pH transition. Certain Candida species (e.g., Candida albicans) for instance, are believed to produce metabolites or other byproducts that alter the pH of the growth medium to about 6.6. Thus, pH-sensitive colorants that undergo a change in pH at or near this level may be used in the present invention. Phenol Red (i.e., phenolsulfonephthalein), for example, may be particularly suitable in that it exhibits a transition from yellow to red over a pH range of about 6.6 to 8.0.

Other phthalein colorants, however, may also be used in the present invention to indicate the presence of Candida. Derivatives of Phenol Red, for instance, may be employed, such as those substituted with chloro, bromo, methyl, sodium carboxylate, carboxylic acid, hydroxyl and amine functional groups. Exemplary substituted Phenol Red compounds include, for instance, Chlorophenol Red, Metacresol Purple (meta-cresolsulfonephthalein), Cresol Red (ortho-cresolsulfonephthalein), Pyrocatecol Violet (pyrocatecolsulfonephthalein), Chlorophenol Red (3′,3″-dichlorophenolsulfonephthalein), Xylenol Blue (the sodium salt of para-xylenolsulfonephthalein), Xylenol Orange, Mordant Blue 3 (C.I. 43820), 3,4,5,6-tetrabromophenolsulfonephthalein, Bromoxylenol Blue, Bromophenol Blue (3′,3″,5′,5″-tetrabromophenolsulfonephthalein), Bromochlorophenol Blue (the sodium salt of dibromo-5′,5″-dichlorophenolsulfonephthalein), Bromocresol Purple (5′,5″-dibromo-ortho-cresolsulfonephthalein), Bromocresol Green (3′,3″,5′,5″-tetrabromo-ortho-cresolsulfonephthalein), and so forth. Still other suitable phthalein colorants are well known in the art, and may include Bromothymol Blue, Thymol Blue, Bromocresol Purple, thymolphthalein, and phenolphthalein (a common component of universal indicators). For example, Chlorophenol Red exhibits a transition from yellow to red over a pH range of about 4.8 to 6.4; Bromothymol Blue exhibits a transition from yellow to blue over a pH range of about 6.0 to 7.6; thymolphthalein exhibits a transition from colorless to blue over a pH range of about 9.4 to 10.6; phenolphthalein exhibits a transition from colorless to pink over a pH range of about 8.2 to 10.0; Thymol Blue exhibits a first transition from red to yellow over a pH range of about 1.2 to 2.8 and a second transition from yellow to pH over a pH range of 8.0 to 9.6; Bromophenol Blue exhibits a transition from yellow to violet over a pH range of about 3.0 to 4.6; Bromocresol Green exhibits a transition from yellow to blue over a pH range of about 3.8 to 5.4; and Bromocresol Purple exhibits a transition from yellow to violet over a pH of about 5.2 to 6.8.

In another embodiment, the off-gas produced by microorganisms can be detected to indicate the presence of and identify the specific microbes via a vivid change in color of the chemical indicator in the temporary tattoo. Each class of microorganism gives off gases that are quite unique to that class of microbe, much in the same way that they also smell differently (e.g., Petri dishes containing live culture of E. coli has a distinctive sour amine smell whereas Candida albicans smells like fresh bread). This reaction would cause a change in color that would be vivid enough for the user to clearly see the change (e.g. typically ΔE≧5). The following table shows the successful results of commonly found microbes that would be important to detect via the tattoo application.

TABLE 1
Color Changing Dyes Identified for Unique Microbial Gaseous
Chemicals.

| | | Color Changing Dye |
|---|---|---|
| | Unique Gaseous | Sensitive to Specific |
| Microbe Type | Compound Identified | Chemical |
| E. coli | Indole | Dimethylaminocinnamaldehyde |
| S. aureus | 2-Acetyl thiazole | 2,4-dinitrophenylhydrazine |
| P. aurignosa | Methyl 2-methyl-2- | Potassium permanganate |
| | butenoate | |
| Candida | Iso-amyl alcohol | Ammonium dichromate |
| albicans | | |
| Salmonella | 3,5-dimethylpyrazine | Potassium permanganate |

C. Amine Chemical Indicators

In one embodiment, the presence of amine compounds can be detected by the temporary tattoos of the present invention. Amine compounds are associated with certain types of infections, when secreted from the body. For example, both bacterial vaginosis and trichomoniasis produce tell-tale amine odors that are typical of vaginal yeast infection or vulvovaginal candidiasis. In bacterial vaginosis, several members of anaerobic bacteria, prevotella, bacteroides, mobiluncus, and peptococcus, are present in large numbers in the vagina. Some of these organisms produce metabolic products such as amines, including trimethyl amine, putrescine, cadaverine, and tyramine which are is responsible for the odor noticed by the affected patients. A “Whiff test” is routinely conducted for amine odors where enhanced odor is generated by adding strong alkali to the sample, however, this test must be professionally performed and the use of strong alkali is not applicable to the skin due to the caustic nature of the chemical.

As such, the temporary tattoo of the present invention can be applied to a wearer in an area near the crotch region of the body to enable the detection of amine compounds. For instance, the temporary tattoo can include an amine sensitive chemichromic dye to indicate the presence of amines in vaginal odor and thus to signal a possible infection.

One class of chemichromic dyes that is particularly useful is arylmethane dyes, such as diarylmethanes, triarylmethanes, and the like. In some embodiments, the side chains of the triarylmethanes can be independently selected from substituted and unsubstituted aryl groups, such as phenyl, naphthyl, anthracenyl, etc. The aryl groups may, for example, be substituted with functional groups, such as amino, hydroxyl, carbonyl, carboxyl, sulfonic, alkyl, and/or other known functional groups. When contacted with the tattoo, the amino group of the amine (e.g., ammonia, diamines, and/or tertiary amines) reacts with the central carbon atom of the chemical indicator. The addition of the amino group causes the chemical indicator to undergo a change in color.

One particular example of a suitable triarylmethane dye is pararosanilin (also known as “basic fuchsin” or “magenta 0”) and analogs thereof, such as rosanilin (“magenta I”), magenta II, new fuchsin (“magenta III”), methyl violet 2B, methyl violet 6B, methyl violet 10B (“crystal violet”), methyl green, ethyl green, acid fuchsin, and so forth. Pararosanilin shifts from a red color to colorless (i.e., white) upon reaction with an amine. Pararosanilin contains three phenylamine groups (i.e., amino-substituted aryl groups).

In some cases, triarylmethane dyes may be formed by converting a leuco base to a colorless carbinol and then treating the carbinol with an acid to oxidize the carbinol and form the dye. Thus, for example, pararosanilin may be derived by reacting the carbinol form of pararosanilin (“pararosaniline base”) with an acid, such as, but not limited to, sulfonic acids, phosphoric acids, hydrochloric acid, and so forth.

Another example of a suitable triarylmethane dye is alpha-naphtholbenzein and analogs thereof. Alpha-naphtholbenzein turns from an orange/red color to a gray/black color upon reaction with an amine. Alpha-naphtholbenzein contains a hydroxyl-substituted naphthyl group, a carbonyl-substituted naphthyl group, and a phenyl group.

Still another example of a suitable triarylmethane dye is naphthocrome green and analogs thereof. Naphthocrome green turns from a pale yellow color to a blue/green color upon reaction with an amine. Similar to alpha-naphtholbenzein, naphthocrome green contains a hydroxyl-substituted naphthyl group, a carbonyl-substituted naphthyl group, and a phenyl group. However, each naphthyl group is also substituted with a sodium carboxyl.

As indicated above, diarylmethanes may also be used. One example of such a diarylmethane is 4,4′-bis(dimethylamino)benzhydrol (also known as “Michler's hydrol”).

Still other examples include analogs of Michler's hydrol, such as Michler's hydrol leucobenzotriazole, Michier's hydrol leucomorpholine, Michler's hydrol leucobenzenesulfonamide, and so forth, as well as other diarylmethanes, such as malachite green leuco, malachite green carbinol, sodium 2,6-dichloroindopheno-late, rhodamine lactam, crystal violet lactone, and crystal violet leuco.

Several suitable chemical indicators for use in the temporary tattoos of the present invention are disclosed in U.S. Patent Application No. 2005/0124072, which is incorporated herein by reference.

D. Other Compositions

Of course, other compositions may be present within the temporary tattoo along with the chemical indicator, as long as these compositions do not affect the ability of the chemical indicator to detect the presence of the targeted analyte. For example, surfactants may be utilized help enhance the contrast between different indicators. Particularly desired surfactants are nonionic surfactants, such as ethoxylated alkylphenols, ethoxylated and propoxylated fatty alcohols, ethylene oxide-propylene oxide block copolymers, ethoxylated esters of fatty (C8-C18) acids, condensation products of ethylene oxide with long chain amines or amides, condensation products of ethylene oxide with alcohols, acetylenic diols, and mixtures thereof. Various specific examples of suitable nonionic surfactants include, but are not limited to, methyl gluceth-10, PEG-20 methyl glucose distearate, PEG-20 methyl glucose sesquistearate, C11-15 pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C6-C22) alcohol, including 3 to 20 ethylene oxide moieties, polyoxyethylene-20 isohexadecyl ether, polyoxyethylene-23 glycerol laurate, polyoxy-ethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxy-ethylene-6-tridecyl ether, laureth-2, laureth-3, laureth-4, PEG-3 castor oil, PEG 600 dioleate, PEG 400 dioleate, and mixtures thereof. Commercially available nonionic surfactants may include the SURFYNOL® range of acetylenic diol surfactants available from Air Products and Chemicals of Allentown, Pa. and the TWEEN® range of polyoxyethylene surfactants available from Fischer Scientific of Pittsburgh, Pa.

A binder may also be employed to facilitate the immobilization of the chemical indicator on the skin of the wearer. For example, water-soluble organic polymers may be employed as binders, such as polysaccharides and derivatives thereof. Polysaccharides are polymers containing repeated carbohydrate units, which may be cationic, anionic, nonionic, and/or amphoteric. In one particular embodiment, the polysaccharide is a nonionic, cationic, anionic, and/or amphoteric cellulosic ether. Suitable nonionic cellulosic ethers may include, but are not limited to, alkyl cellulose ethers, such as methyl cellulose and ethyl cellulose; hydroxyalkyl cellulose ethers, such as hydroxyethyl cellulose, hydroxypropyl cellulose, hydroxypropyl hydroxybutyl cellulose, hydroxyethyl hydroxypropyl cellulose, hydroxyethyl hydroxybutyl cellulose and hydroxyethyl hydroxypropyl hydroxybutyl cellulose; alkyl hydroxyalkyl cellulose ethers, such as methyl hydroxyethyl cellulose, methyl hydroxypropyl cellulose, ethyl hydroxyethyl cellulose, ethyl hydroxypropyl cellulose, methyl ethyl hydroxyethyl cellulose and methyl ethyl hydroxypropyl cellulose; and so forth.

### IV. Detection of Color Chance

The degree to which an indicator changes color may be determined either visually or using instrumentation. In its simplest form, the temporary tattoo can change colors upon contact with the analyte in a manner that is readily visible to the wearer and/or caregiver without the need for any visual aid or other instrumentation. Typically a visual change in color of >5 ΔE most humans can visually see changes in color (color 1 to color 2) or increase or decrease of the same shade of color. In one embodiment, for instance, the display can be worn as a on the wrist of the wearer or caregiver.

However, in other embodiments, the color intensity can be measured with an optical reader. The actual configuration and structure of the optical reader may generally vary as is readily understood by those skilled in the art. Typically, the optical reader contains an illumination source that is capable of emitting electromagnetic radiation and a detector that is capable of registering a signal (e.g., transmitted or reflected light). The illumination source may be any device known in the art that is capable of providing electromagnetic radiation, such as light in the visible or near-visible range (e.g., infrared or ultraviolet light). For example, suitable illumination sources that may be used in the present invention include, but are not limited to, light emitting diodes (LED), flashlamps, cold-cathode fluorescent lamps, electroluminescent lamps, and so forth. The illumination may be multiplexed and/or collimated. In some cases, the illumination may be pulsed to reduce any background interference. Further, illumination may be continuous or may combine continuous wave (CW) and pulsed illumination where multiple illumination beams are multiplexed (e.g., a pulsed beam is multiplexed with a CW beam), permitting signal discrimination between a signal induced by the CW source and a signal induced by the pulsed source. For example, in some embodiments, LEDs (e.g., aluminum gallium arsenide red diodes, gallium phosphide green diodes, gallium arsenide phosphide green diodes, or indium gallium nitride violet/blue/ultraviolet (UV) diodes) are used as the pulsed illumination source.

The detector may generally be any device known in the art that is capable of sensing a signal. For instance, the detector may be an electronic imaging detector that is configured for spatial discrimination. Some examples of such electronic imaging sensors include high speed, linear charge-coupled devices (CCD), charge-injection devices (CID), complementary-metal-oxide-semiconductor (CMOS) devices, and so forth. Such image detectors, for instance, are generally two-dimensional arrays of electronic light sensors, although linear imaging detectors (e.g., linear CCD detectors) that include a single line of detector pixels or light sensors, such as, for example, those used for scanning images, may also be used. Each array includes a set of known, unique positions that may be referred to as “addresses.” Each address in an image detector is occupied by a sensor that covers an area (e.g., an area typically shaped as a box or a rectangle). This area is generally referred to as a “pixel” or pixel area. A detector pixel, for instance, may be a CCD, CID, or a CMOS sensor, or any other device or sensor that detects or measures light. The size of detector pixels may vary widely, and may in some cases have a diameter or length as low as 0.2 micrometers.

In other embodiments, the detector may be a light sensor that lacks spatial discrimination capabilities. For instance, examples of such light sensors may include photomultiplier devices, photodiodes, such as avalanche photodiodes or silicon photodiodes, and so forth. Silicon photodiodes are sometimes advantageous in that they are inexpensive, sensitive, capable of high-speed operation (short risetime/high bandwidth), and easily integrated into most other semiconductor technology and monolithic circuitry. In addition, silicon photodiodes are physically small, which enables them to be readily incorporated into various types of detection systems. If silicon photodiodes are used, then the wavelength range of the emitted signal may be within their range of sensitivity, which is 400 to 1100 nanometers.

Optical readers may generally employ any known detection technique, including, for instance, luminescence (e.g., fluorescence, phosphorescence, etc.), absorbance (e.g., fluorescent or non-fluorescent), diffraction, etc. In one particular embodiment of the present, the optical reader measures color intensity as a function of absorbance.

The optical reader can be, in one embodiment, utilized as a portable monitor to be easily monitored by the wearer and/or caregiver. For example, the optical reader can be interfaced with the temporary tattoo and can provide a signal (e.g., via a wired connection or wireless connection) to a display unit. The display unit can then alert the wearer and/or caregiver that the presence of the targeted analyte has (or has not) been detected.

### EXAMPLES

### Example 1

### Ferric Chloride

A solution of ferric chloride in isopropanol (50 mg/ml) was shown to instantly turn from a yellow color to a dark blue/black color when mixed with a drop (10 mg/ml) of 4-t-butyl catechol (Aldrich Chemic Company, Milwaukee Wis.). This color change was also observed after applying the ferric chloride solution to Avery label, plain paper and coform wipe stock to give a yellow stain. The dye was allowed to air dry before exposing the items to a drop of the t-butylcatechol solution. On contact of the catechol solution with the items the yellow color was instantly converted to the blue/black color. Thus, it can be seen that the ferric chloride treated articles can be used as visual indicators of urushiol contact and contamination.

### Example 2

### 4-Amino-antipyrine

A solution of 4-amino-antipyrine (Aldrich Chemical Company, Milwaukee Wis.) in isopropanol (50 mg/ml) with a drop of sodium hydroxide (0.5N) was shown to change from a colorless solution to a deep red in color when mixed with a drop of 4-t-butylcatachol (10 mg/ml) in isopropanol. This color change was also observed when the aminoantipyrine solution was applied to Avery label stock, plain paper and coform wipe stock to give a colorless coating which was allowed to air dry. To the treated items a drop of the 4-t-butylcatechol was applied resulting in a quick development of the deep red color.

### Example 3

### Testing with Actual Poison Ivy, Ferric Chloride Indicator

Actual samples of real poison ivy were carefully collected in a green space park in Decatur, Ga. (Mason Mill Park green space). The triple leaf sections were cut and placed in plastic bags (Zip-loc® S. C. Johnson & Son, Inc.) while wearing nitrile gloves. Approximately 4 sections were placed in each bag. To the bag was placed one Avery label, treated with ferric chloride as described in Example 1, and the bag shaken for 3 minutes to expose the label to the leaves. Parts of the yellow spot on the label were observed to instantly turn blue/black indicating exposure to the poison ivy's urushiol oil. Thus, real-world testing of these indicator stickers was successful.

### Example 4

### Reichart's Dye

Reichart's dye (Aldrich Chemical Co., Milwaukee Wis.) in isopropanol solution (50 mg/ml) was coated onto a temporary tattoo and allowed to dry. The tattoo was then applied to the top of a volunteer's hand. Next, the skin area having the tattoo had a small amount of yogurt culture (200 μl) applied by means of a small paint brush. Yogurt has a live culture of lactobacius at log5 cfu. When the dye of the tattoo was contacted with the yogurt the purple color turns colorless or white giving the user a vivid indication to them of coming in contact with microbial contamination and the hand was now contaminated.

### Example 5

### Off-Gas of Microbes

The specific unique gas or vapor given off by a series of bacteria and yeasts were identified by GC Headspace followed by mass spectroscopic analysis (i.e., “GC-MS”). Once the characteristic chemical for the microbe was identified a colored chemical compound that would react with this off-gas was then researched.

TABLE 2
Visual Color Change Triggered by Unique Bacteria Model Odors

| Microbial - Odor Compound | Initial Color | Final Color |
|---|---|---|
| E. coli - Indole odor | Light pink | Deep purple |
| S. aureus - 2-Acetyl thiazole odor | Light yellow | Orange |
| Candida albicans - iso-amyl alcohol | Orange | Green |
| Salmonella - 3,5-dimethylpyrazine | Light pink | Deep purple |
| P. aurignosa - methyl 2-methyl-2-butenoate | Purple | Brown |

The microbe active compound was coated onto the film surface and allowed to dry. A 1 cm×1 cm square of the coated film was attached to the lid of a 20 ml glass container in order that it would be suspended on the inside of the container when the lid was screwed on. Into the bottom of the glass container was placed 100 μl of microbial suspension at a typical concentration of log5 cfu. The lid was then placed on the container allowing the test strip to hang over, but not touch, the microbial liquid. Within seconds the color of the test strip changed indicating that the specific microbes were present to the observer.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. A temporary tattoo decal for application to a wearer's skin and configured to detect the presence of an analyte, the temporary tattoo decal comprising
a base paper;
a water-soluble slip layer applied to the base paper;
a temporary tattoo applied to the water-soluble release layer on the base paper in the form of an image, wherein the temporary tattoo comprises a chemical indicator configured to change color upon contact with the analyte;
an adhesive layer overlying the temporary tattoo; and
a protective sheet overlying the adhesive layer.

2. A temporary tattoo decal as in claim 1, wherein the chemical indicator is substantially transparent until contact with the analyte, and wherein the chemical indicator becomes visible upon contact with the analyte.

3. A temporary tattoo decal as in claim 1, wherein the chemical indicator changes from a first visible color to a second visible color upon contact with the analyte.

4. A temporary tattoo decal as in claim 1, wherein the chemical indicator becomes substantially transparent upon contact with the analyte.

5. A temporary tattoo decal as in claim 1, wherein the analyte comprises a microorganism.

6. A temporary tattoo decal as in claim 5, wherein the chemical indicator comprises a solvatochromatic indicator.

7. A temporary tattoo decal as in claim 6, wherein the solvatochromatic indicator comprises a merocyanine indicator.

8. A temporary tattoo decal as in claim 6, wherein the solvatochromatic indicator comprises a permanent zwitterionic form having formal positive and negative charges contained within a contiguous π-electron system.

9. A temporary tattoo decal as in claim 1, wherein the chemical indicator comprises 2,6-dibromoquinone-4-chlorimide, ferric chloride, 4-amino-antipyrine or combinations thereof.

10. A temporary tattoo decal as in claim 1, wherein the chemical indicator comprises 4,4′-bis(dimethylamino)-benzhydrol.

11. A temporary tattoo decal as in claim 1, wherein the chemical indicator comprises a pH-sensitive indicator configured to detect a change in pH.

12. A temporary tattoo decal as in claim 1, wherein the chemical indicator is configured to detect the presence of yeast.

13. A temporary tattoo decal as in claim 1, wherein the chemical indicator is configured to detect specific off-gases generated by microbes.

14. A temporary tattoo decal as in claim 1, wherein the chemical indicator is configured to detect the presence of bacteria, mold, yeast or fungi.

15. A method of detecting the presence of an analyte, the method comprising
applying a temporary tattoo to skin of a wearer, wherein the temporary tattoo forms an image and comprises a chemical indicator configured to change color upon contact with the analyte; and
monitoring the temporary tattoo to determine if a color change has occurred.

16. A method as in claim 15, wherein the applying the temporary tattoo to the skin of a wearer comprises
peeling a protective sheet from a temporary tattoo decal to expose an adhesive layer, wherein the temporary tattoo decal comprises a base paper, a water-soluble slip layer applied to the base paper, a temporary tattoo applied to the water-soluble release layer on the base paper in the form of an image, the adhesive layer overlying the temporary tattoo, and the protective sheet overlying the adhesive layer;
positioning the exposed adhesive layer adjacent the skin of the wearer;
moistening the base paper with a solvent to disperse the water-soluble slip layer; and
removing the base paper to leave the temporary tattoo on the skin.

17. A method as in claim 15, wherein the chemical indicator is substantially transparent until contact with the analyte, and wherein the chemical indicator becomes visible upon contact with the analyte.

18. A method as in claim 15, wherein the chemical indicator changes from a first visible color to a second visible color upon contact with the analyte.

19. A method as in claim 15, wherein the chemical indicator becomes substantially transparent upon contact with the analyte.

20. A method as in claim 15, wherein the analyte comprises a microorganism.
